(19) United States
(12) Patent Application Publication
(10) Pub. No.: US 2005/0065815 A1
(43) Pub. Date: Mar. 24, 2005

(54) Information management system and method for an implantable medical device

(76) Inventors: Scott Thomas Mazar, Inver Grove Heights, MN (US); Bruce H. Kenknight, Maple Grove, MN (US)

Correspondence Address: Merchant & Gould P.C., P.O. Box 2903, Minneapolis, MN, 55402-0903, US

(21) Appl. No.: 10/665,792

(22) Filed: Sep. 19, 2003

(51) Int. Cl. G06F 17/60; A61B 5/00

(52) U.S. Cl. 705/2; 607/60; 600/300

(57) ABSTRACT

Systems and methods for an information management system for an implantable medical device are disclosed. A preferred embodiment uses an implantable medical device that is automatically recognized by a publicly accessible information access portal. Some embodiments of a system disclosed herein also can be configured as a component of an Advanced Patient Management System that helps better monitor, predict and manage chronic diseases.

### TECHNICAL FIELD

The present systems and methods relate generally to a Patient Management System and particularly, but not by way of limitation, to such a system that provides patients and clinicians with ubiquitous access to patient health data.

### BACKGROUND

Currently, access to patient health data or information, either by a patient or a clinician, is limited to retrieving such information from a medical information repository like a physician's office or hospital. In almost every instance, this information is not immediately available to the patient. Typically, the patient can only obtain information on his or her health state by first requesting that information through various levels of administrative bureaucracy implemented to insure patient confidentiality. For example, a patient requesting a copy of his medical record usually must make such a request in writing and provide sufficient proof of identity. When the patient finally receives the medical file, the information therein is current only up to his last medical visit. In addition, the patient does not have any means to personally amend the medical record to reflect a current state of health.

Computer technologies and other electronic tools partially solve the problem of quick and efficient access to medical records. For example, the patient's medical record can be presented in electronic form for easy transmission and manipulation by the patient or clinician. However, access to electronic medical record information is still limited by customary means of identifying the patient or other requester to insure confidentiality.

However, it is now possible to automatically identify a person using computer technologies to recognize a unique physical or biometric characteristic of the person. For example, fingerprint identification has long been used to distinguish individuals. Even now, banking institutions routinely obtain a thumbprint impression from a person seeking to cash a check or other negotiable instrument. However, confirming the identity of a person through fingerprint recognition is often done after the fact. That problem is partially solved by computers that have been configured to rapidly identify a fingerprint contained within a fingerprint database.

Banks and other institutions also employ the use of unique personal identification numbers (PINs) to identify people. However, a person must typically carry a plastic card containing preliminary information embedded on a magnetic strip and memorize the PIN to initiate the identification process. However, memorization may be challenging to the elderly or others suffering from mental impairment. In addition, if another person has access to the PIN, the typical recognition system is incapable of automatically and instantaneously distinguishing a person authorized to use the PIN from a person who is not.

Security access cards also provide a measure of personal identification. However, as with PINs, access card readers or scanners typically cannot identify the person if the security card is in the possession of another and thus, are not a reliable means of personal identification. However, the proximity recognition technology employed by these systems may have value in identifying a person if such technology is embedded or implanted in the person.

Voice recognition systems also identify people. However, voice recognition systems, when used to permit access to confidential information, may improperly grant or deny access due to physiological changes to the speech pattern. For example, a patient's speech pattern may be altered and unrecognizable during a bout with a common cold or other respiratory ailment. Thus, while promising, voice recognition systems may not be discriminating enough to precisely identify a person under all circumstances.

Because of increasing concerns about terrorism, facial-identification systems are receiving more attention. Current facial-identification systems require specialized video-capturing devices that are adapted to communicate with extensive computer databases to match facial profiles captured by the video device to images in the database. However, the expense and untested reliability of this technology may be prohibitive.

Finally, retinal scans offer promise as an accurate way to identify people. The retina is the surface inside the back of the eye. The blood vessels on a person's retina have been shown to be a unique to each person much like a fingerprint. To use a retinal scanner for identification purposes, the user places his eye relatively close (between 1 and 2 inches) to a reader and focuses on a rotating light. To enroll a person into a retinal scan database, the retina is scanned multiple times. Thereafter, to identify a person in the database, only a single scan is needed.

By coupling a reliable personal identification system to medical record access, a person authorized to obtain otherwise confidential medical information will have greater access to the information. Indeed, because a patient's medical information can be transmitted electronically, there are few limits on a patient's or clinician's ability to obtain medical information at any place or any time. In addition, once a proper personal identification has been made to insure patient confidentiality, a patient or clinician will have the ability to amend the medical record to reflect a patient's current objective or subjective health status. In this way, accurate assessments of patient health can be accomplished with much greater frequency than the conventional way of assessing patient health by visiting a physician's office or a hospital, and the patient or clinician can more quickly observe trends in patient health. The efficiency of such a system should give patients and clinicians the ability to more accurately predict and preempt states of health degradation in addition to providing positive reinforcement of improvements in health.

In addition to confidential medical record information, the system may also grant access to non-confidential information of interest to the person. For example, the system may allow a user to configure the information access portal with content of particular interest to the person much like the My Yahoo!™ Internet-based system. Such content may include weather information, news reports, economic information and a daily horoscope.

Thus, for these and other reasons, there is a need for a Patient Management System comprising components capable of accurately identifying a person authorized to receive confidential medical information and other non-confidential information and components to convey that information through an access portal easily accessible by the patient or clinician. In this way, the Patient Management System will lower the cost of medical care by placing greater emphasis on disease prevention rather than treating acute episodes of disease.

### SUMMARY

According to one aspect of the invention, there is provided an information management system comprising an implantable medical device adapted to sense and transmit patient health data; a patient management system adapted to store and analyze patient health data; a recognition module adapted to uniquely identify a person authorized to access patient health data; and an information access portal adapted to convey patient health data and other information to an authorized, uniquely identified person. As used herein, “patient data” or “patient health data” includes physiometry or physiometric data. Physiometry data is a measurement of a person's or patient's physiological or psychological state. Also, as used herein, a “clinician” can be a physician, physician assistant (PA), nurse, medical technologist, or any other patient health care provider.

The patient management system may be configured as an Advanced Patient Management (“APM”) system and the implantable medical device may comprise a component of the APM system. The APM system may include clinically derived algorithms to analyze patient data and be remotely stationed from the information access portal.

The recognition module may comprise a fingerprint recognition system, a security access card system, a voice recognition system, a facial-identification system, a retinal scan recognition system or a proximity recognition system embedded in an implantable medical device that uniquely identifies the person with the device. In a preferred embodiment, the recognition module automatically recognizes the proximity recognition system embedded in the implantable medical device as the person approaches an information access portal comprising the recognition module. A proximity recognition system within the context of the invention includes systems that are capable of uniquely identifying an implantable medical device and, accordingly, the person within whom the device has been implanted.

The information access portal is adapted for easy and convenient access by either a patient or a clinician. The access portal may comprise a home or portable interface system like a personal computing device, a kiosk of the type commonly found in a shopping mall or an automatic teller machine/ATM-like system. In a preferred embodiment, the access portal is publicly available and commonly found, much like an ATM, to provide ubiquitous access to patient data and other information.

Once a person gains access to the system, the system is capable of conveying the person's physiometric data via a multi-media presentation. The system also is adapted to allow the person to enter current physiometric data into the system. The system is further adapted to allow the person to compare the person's data to a population of persons who may share a similar health characteristic with the person.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention, which is set forth in the following claims.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic/block diagram illustrating generally, among other things, one embodiment of an information management system for an implantable medical device.

FIG. 2 is a schematic/block diagram illustrating generally, among other things, the APM configuration of the information management system for an implantable medical device.

FIG. 3 is a schematic/block diagram illustrating generally, among other things, the recognition component of the information management system for an implantable medical device.

FIG. 4 is a schematic/block diagram illustrating generally, among other things, the information access portal adapted to convey patient health data and other information to an authorized, uniquely identified person.

FIG. 5 is a flow chart illustrating generally, among other things, a method for an information management system for an implantable medical device.

### DETAILED DESCRIPTION

In the following detailed description, reference is made to the accompanying drawings that form a part hereof, and in which are shown by way of illustration specific embodiments or examples. These embodiments may be combined, other embodiments may be utilized, and structural, logical and electrical changes may be made without departing from the spirit and scope of the present invention. The following detailed description is, therefore, not to be taken in a limiting sense, and the scope of the present invention is defined by the appended claims and their equivalents.

The present system is described with respect to an information management system for an implantable medical device adapted to provide ubiquitous access to patient data and other information through an information access portal that automatically identifies a person authorized to receive the data or information. The system may further be adapted to receive physiometry data from a patient or clinician. Such ubiquitous access to patient data and the ability to input physiometry data allows the system to augment a preventive health regimen by allowing the patient or clinician to readily observed trends in patient health.

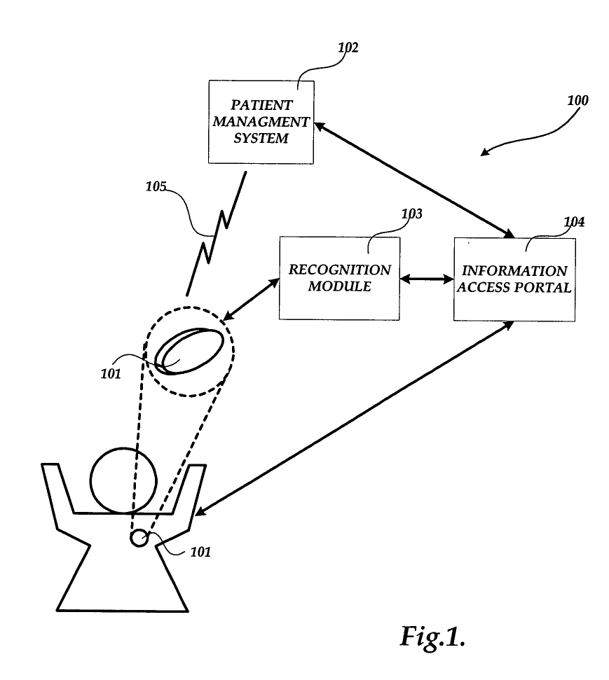

FIG. 1 is a schematic/block diagram illustrating generally an embodiment of an information management system 100 for an implantable medical device capable of providing ubiquitous access to patient data or other information. The system comprises an implantable medical device 101 adapted to sense and transmit patient health data; a patient management system 102 adapted to store and analyze patient health data; a recognition module 103 adapted to uniquely identify a person authorized to access patient health data; and an information access portal 104 adapted to convey patient health data and other information to an identified person authorized to receive the information.

As further shown in FIG. 1, the implantable medical device 101 may comprise an implantable device like a pacemaker that is adapted to sense and electronically transmit 105 patient data to the patient management system 102. By way of non-limiting example only, patient data may comprise cardiovascular data, electro-chemical data, blood chemistry data, temperature data, wedge pressure data, oxygen saturation data, weight data, subjective well-being input data, blood pressure data, electrocardiogram (“ECG/EKG”) data or any other physiological parameter suitable for measurement by the sensing component of the implantable medical device. Collectively, physiometry data includes such objective and subjective patient data. As further shown in FIG. 1, the implantable medical device 101 may wirelessly transmit 105 patient data to the patient management system 102. Wired data transmission technologies may also be employed. The patient management system 102 is adapted to store and analyze physiometry data. When configured as an Advanced Patient Management system, the patient management system 102 is analytically robust to embody features of artificial intelligence.

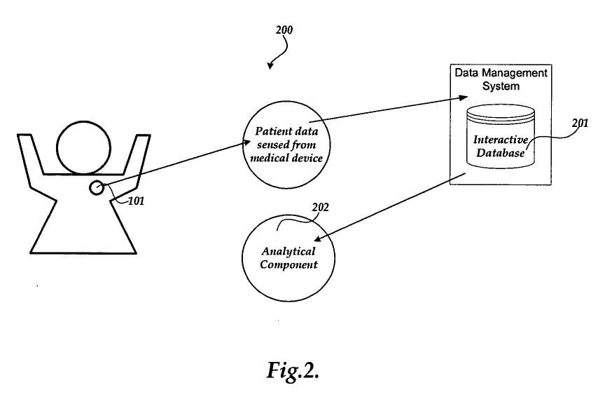

APM is a system that helps patients, their physicians and their families to better monitor, predict and manage chronic diseases. In the embodiment shown in FIG. 2, the APM system 200 comprises three primary components: 1) an implantable medical device 101 with sensors adapted to monitor patient data, 2) a Data Management System (“DMS”), which in this embodiment is shown as interactive database 201 and 3) an analytical component 202 adapted to analyze and correlate data from the DMS. APM is designed to support physicians and other clinicians in using a variety of different devices, patient-specific and non-specific data, along with medication therapy, to provide the best possible care to patients. The analytical component 202 of APM may include the use of clinically derived algorithms that reflect or embody a standard of medical care. Such standards of medical care can reflect the institutional practices and methodologies of institutions like, by way of non-limiting example only, the Cleveland Clinic, the Mayo Clinic or the Kaiser Permanente system, that have been reduced to algorithmic expression. Currently, implanted devices often provide only limited sensing, analysis and therapy to patients. APM moves the device from a reactive mode into a predictive one that allows a clinician to use APM to predict patient health.

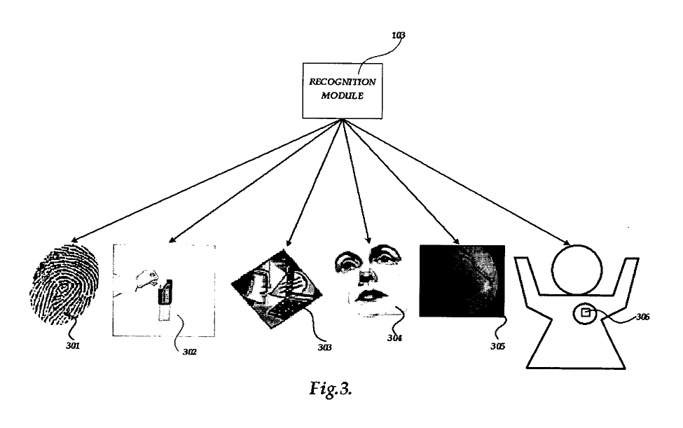

FIG. 3 is a schematic/block diagram illustrating generally, among other things, the recognition component of the information management system for an implantable medical device. The recognition module may comprise a fingerprint recognition system 301. In this embodiment, the recognition module comprises a fingerprint database comprising all the individuals, either patients or clinicians, authorized to view patient health data.

In any of the embodiments described herein, a person other than a clinician may be authorized to view another's patient health data. By way of non-limiting example only, an elderly parent may authorize a daughter to view her patient health data. This relieves the patient of that burden and may be especially important if the patient is infirm or non-ambulatory.

As further shown in FIG. 3, the recognition component may comprise a security access card system 302. In this embodiment, the patient, clinician or other authorized person gains access to the information access portal by swiping or presenting the card at the portal site. After the recognition component confirms the validity of the access card and identifies the authorized person, the person can access the portal to obtain and enter physiometric data. In this embodiment, a security access card may also be of the type that is read or scanned, much in the way bar codes are typically scanned in grocery stores.

The recognition component may comprise a voice recognition system 303 as shown in FIG. 3. Typically, a person must first train the voice recognition system to recognize his or her voice. Afterwards, the recognition component is capable of distinguishing the person's speech patterns and/or inflections from others. Once distinguished, the information access portal 104 allows the identified person to access the system 100.

The recognition component may also comprise a facial-identification system 304 as shown in FIG. 3. In this embodiment, the patient, clinician or other authorized person gains access to the information access portal by looking at a device or camera that captures an image of the person. The image can then be electronically and automatically matched to a database of stored images to determine if the person is authorized to access the access portal.

As further shown in FIG. 3, the recognition component may comprise a retinal scan recognition system 305. In this embodiment, a person seeking access to the information access portal may look into an eyepiece adapted to scan the person's retina. If the scanned retina matches a retina of a person previously enrolled in the system, the person seeking access is automatically granted access to the access portal.

Finally, the recognition component may comprise a proximity recognition system 306 embedded within the implantable medical device that uniquely identifies the person with the device as shown in FIG. 3. In this embodiment, the person seeking access to the information access portal need only approach the portal (should the portal comprise the recognition module) to a distance that allows the recognition module to communicate with the proximity recognition system. If the recognition module recognizes the proximity system embedded within the device, the person seeking access is automatically granted access to the access portal. Once access has been granted, the person would be authorized to access to his physiometric data and other information and/or the physiometric data or other information of another person.

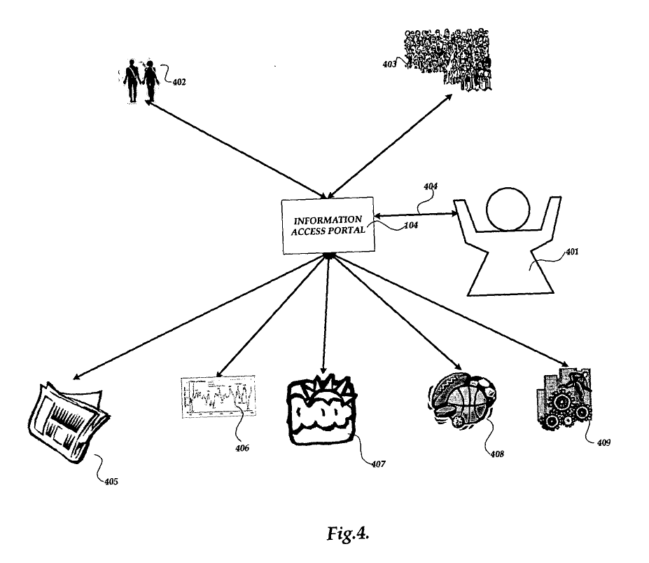

FIG. 4 is a schematic/block diagram illustrating generally, among other things, the information access portal adapted to convey patient health data and other information to an authorized, uniquely identified person. Once a person 401 gains access to the information access portal 104, the person is entitled to view his or her physiometric data 402. In addition, an authorized person may be able to view health population data 403 and compare the person's health state to the population. The system is adapted to allow the person 401 to configure the health population data parameters. By way of non-limiting example only, a person 401, whether a patient or clinician, may select and compare a population of patients of similar age and weight to the patient or, instead, select population data reflecting standard physiometric parameters. In this way, a patient will have a point of reference for his health state that extends beyond personal, physiometric information. The person, whether a patient or clinician, may also be entitled to enter 404 physiometric data into the system. By allowing a patient or clinician to continually update the system with current physiometric data, the system has the greatest potential to recognize and report relevant trends in patient health.

The information access portal may be configured to disclose only certain information to the identified person. By way of non-limiting example only, if the recognition system grants access to a patient, the patient might have full access to all of his or her confidential or non-confidential information. If the recognition system grants access to the patient's physician, for example, the system may only report the patient's medical information. However, medical information presented to the physician or other clinician may be presented in greater detail than medical information presented to the patient. In addition, if the recognition system authorizes another person to access a patient's information, like for example a family member, the system may only report summary medical information and not report non-confidential, personal information.

In addition to physiometric data, as shown in FIG. 4, the patient may have access to other information unrelated to health data. By way of non-limiting example only, such other information may comprise reports of current events 405, stock prices 406, weather 407, sports 408, economic 409 and other information of interest to the person. The system may further be adapted to allow the user to configure the presentation of physiometric or other information. Information may be conveyed in multi-media format. Such formats include, but are not limited to, audio, video and tactile displays.

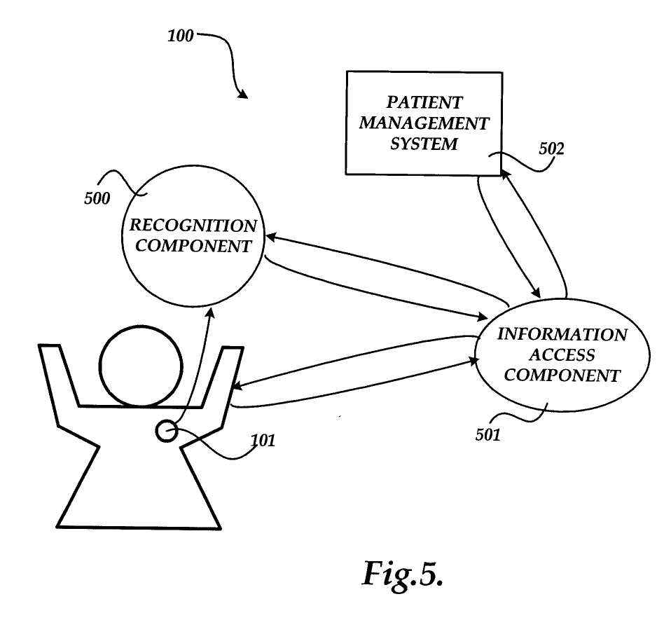

FIG. 5 is a flow chart illustrating generally, among other things, a method for an information management system for an implantable medical device. As shown in FIG. 5, a patient with an implantable medical device 101 approaches a publicly accessible kiosk comprising a recognition component 500. The medical device 101 includes a proximity recognition system adapted to be recognized by the recognition component 500, which automatically identifies and grants the patient access to the information access component 501 of the system 100. The information access component 501 is in electronic communication with the patient management system 502, which comprises the analysis and data storage component of the system 100. The patient management system 502 transmits physiometric data and other information to the information access component 501, which in turn conveys the data and information to the patient. A convenient way to convey the information is via a multi-media display. The display may comprise video, audio and tactile displays of the patient's static or trended physiometric data or a population's static or trended physiometric data either alone or in comparison to the patient's data. Static data or information is best understood as snapshot of patent health. Trended data or information is best understood as a series of patient health snapshots compiled in such a way that trends in patient health can be quickly and easily observed or understood. By way of non-limiting example only, such a trend may be represented by a two-dimensional graphical display of daily blood pressure readings. The information access component 501 also may display other, non health-related information such as world economic news, weather or current events. The information access component 501 is further adapted to allow the patient to input physiometric data into the system 100 to enhance the accuracy of the static or trended physiometric data. Input data is transmitted to the patient management system 502 for inclusion in its databases for future access.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including,” “includes” and “in which” are used as the plain-English equivalents of the respective terms “comprising,” “comprises” and “wherein.”

1. An information management system comprising:
a. an implantable medical device adapted to sense and transmit patient health data;
b. a patient management system adapted to store and analyze patient health data;
c. a recognition module adapted to uniquely identify a person authorized to access patient health data; and
d. an information access portal adapted to convey patient health data and other information to an authorized, uniquely identified person.

2. The system of claim 1, wherein the patient management system comprises an Advanced Patient Management system.

3. The system of claim 2, wherein the implantable medical device comprises a component of the Advanced Patient Management system.

4. The Advanced Patient Management system of claim 2, wherein the system is remote from the information access portal.

5. The system of claim 1, wherein the recognition module comprises a fingerprint recognition system.

6. The system of claim 1, wherein the recognition module comprises a security access card system.

7. The system of claim 1, wherein the recognition module comprises a bar code scanning system.

8. The system of claim 1, wherein the recognition module comprises a voice recognition system.

9. The system of claim 1, wherein the recognition module comprises a facial-identification system.

10. The system of claim 1, wherein the recognition module comprises a retinal scan recognition system.

11. The system of claim 1, wherein the recognition module comprises a proximity recognition system.

12. The proximity recognition system of claim 11, wherein the system comprises a proximity reader and an implantable medical device further comprising an antennae and an integrated circuit.

13. The system of claim 1, wherein the information access portal conveys information.

14. The conveyed information of claim 13, wherein the information is conveyed in a multi-media presentation.

15. The multi-media presentation of claim 14, wherein the presentation comprises audio, video and tactile presentations.

16. The system of claim 1, wherein the information access portal conveys physiometric information.

17. The conveyed physiometric information of claim 16, wherein the information is conveyed in a multi-media presentation.

18. The multi-media presentation of claim 17, wherein the presentation comprises audio, video and tactile presentations.

19. The physiometric information of claim 16, wherein the information comprises static information.

20. The physiometric information of claim 16, wherein the information comprises trended information.

21. The system of claim 1, wherein the information access portal conveys other information.

22. The conveyed other information of claim 21, wherein the information is conveyed in a multi-media presentation.

23. The multi-media presentation of claim 22, wherein the presentation comprises audio, video and tactile presentations.

24. The conveyed other information of claim 21, wherein the person can configure the information.

25. The conveyed other information of claim 21, wherein the conveyed information comprises reports of current events, stock prices, weather, sports, economic and other information.

26. The system of claim 1, wherein the information access portal comprises a home interface system.

27. The home interface system of claim 26, wherein the home interface system comprises a personal computing device.

28. The home interface system of claim 26, wherein the home interface system comprises a portable personal computing device.

29. The system of claim 1, wherein the information access portal comprises a kiosk.

30. The system of claim 1, wherein the information access portal comprises an ATM-like system.

31. The access portal of claim 29, wherein the access portal is publicly available.

32. The access portal of claim 30, wherein the access portal is publicly available.

33. An information management system comprising:
a. an implantable medical device adapted to sense and transmit patient health data comprising a proximity recognition system;
b. an Advanced Patient Management system adapted to store patient health data and analyze patient health data using clinically derived algorithms in a manner consistent with a standard of medical care;
c. a recognition module adapted to uniquely identify the medical device comprising the proximity recognition system and authorize personal access to an information portal; and
d. a publicly accessible information access portal adapted to convey patient health data and other information to an authorized, uniquely identified person in a multi-media presentation.

34. A method for an information management system to convey information to a person comprising the steps of:
a. automatically identifying the person;
b. granting the automatically identified person access to an information access portal;
c. conveying information in the form of physiometric data to the automatically identified person through the information access portal; and
d. conveying other information to the automatically identified person through the information access portal.

35. The method of claim 34, wherein the step of automatically identifying a person comprises the prior step of implanting an identifiable medical device within the person.

36. The method of claim 34, wherein the method comprises the further step of allowing the automatically identified person to enter information into the access portal.

37. The method of claim 34, wherein the step of automatically identifying a person comprises the further step of identifying the person using a fingerprint recognition system.

38. The method of claim 34, wherein the step of automatically identifying a person comprises the further step of identifying the person using a security access card system.

39. The method of claim 34, wherein the step of automatically identifying a person comprises the further step of identifying the person using a bar code scanning system.

40. The method of claim 34, wherein the step of automatically identifying a person comprises the further step of identifying the person using a voice recognition system.

41. The method of claim 34, wherein the step of automatically identifying a person comprises the further step of identifying the person using a facial-identification system.

42. The method of claim 34, wherein the step of automatically identifying a person comprises the further step of identifying the person using a retinal scan recognition system.

43. The method of claim 34, wherein the step of automatically identifying a person comprises the further step of identifying the person using a proximity recognition system.

44. The method of claim 34, wherein the step of automatically identifying a person comprises the further step of identifying a patient.

45. The method of claim 34, wherein the step of automatically identifying a person comprises the further step of identifying a clinician.

46. The method of claim 34, wherein the step of automatically identifying a person comprises the further step of identifying a person authorized to access the access portal.

47. The method of claim 34, wherein the step of conveying physiometric data comprises the further step of conveying static physiometric data.

48. The method of claim 34, wherein the step of conveying physiometric data comprises the further step of conveying trended physiometric data.

49. The method of claim 34, wherein the step of conveying physiometric data comprises the further step of conveying data in a multi-media format.

50. The method of claim 34, wherein the step of conveying other information comprises the further step of conveying information in a multi-media format.

51. The conveyed multi-media format of claim 49, wherein the conveyed format comprises the further step of presenting audio, video and tactical presentations.

52. The conveyed multi-media format of claim 50, wherein the conveyed format comprises the further step of presenting audio, video and tactical presentations.

53. The method of claim 34, wherein the step of conveying physiometric data comprises the further step of conveying data comprising cardiovascular data, electro-chemical data, blood chemistry data, temperature data, wedge pressure data, oxygen saturation data, weight data, subjective well-being data, blood pressure data, EKG data and other physiological or psychological data.

54. The method of claim 34, wherein the step of conveying physiometric data comprises the further step of comparing the physiometric data of the person to the physiometric data from a population of persons.

55. The method of claim 34, wherein the step of conveying physiometric data comprises the further step of comparing the physiometric data of the person to the physiometric data from a population of persons.

56. The step of comparing a person's physiometric data to the physiometric data from a population of persons of claim 55, wherein the step further comprises comparing the physiometric data of the person to the physiometric data from a population of persons with a health profile similar to the person.

57. The step of comparing a person's physiometric data to the physiometric data from a population of persons of claim 55, wherein the step further comprises comparing the physiometric data of the person to the physiometric data from a population of persons, said population physiometric data being selected by the person.

58. The step of comparing a person's physiometric data to the physiometric data from a population of persons of claim 55, wherein the step further comprises comparing the physiometric data of the person to the physiometric data from a population of persons, said population physiometric data being selected by a clinician.

59. The step of comparing a person's physiometric data to the physiometric data from a population of persons of claim 55, wherein the step further comprises comparing the physiometric data of the person to the physiometric data from a population of persons, said population physiometric data being selected by another person so authorized to compare and select said data.

60. The method of claim 34, wherein the step of conveying other data comprises the further step of conveying data comprising reports of current events, stock prices, weather, sports, economic and other information.

61. A method for an information management system to convey information to a person comprising the steps of:
a. automatically identifying a person with an implantable medical device comprising a proximity recognition system;
b. granting the automatically identified person access to a publicly accessible information access portal;
c. conveying information in the form of static or trended physiometric data to the automatically identified person through the information access portal in a multi-media presentation; and
d. conveying other information to the automatically identified person through the information access portal in a multi-media presentation.
